(19) United States
(12) Patent Application Publication
(10) Pub. No.: US 2009/0118682 A1
(43) Pub. Date: May 7, 2009

(54) Reservoir Device With Inspection Aid For Detection Of Drug Condition

(75) Inventors: Steffen Hansen, Hillerod (DK); Per Jeppesen, Bronshoj (DK); Lars Aagaard, Fredensborg (DK); Kirsten Bundgaard-Nielsen, Fredensborg (DK); Anita Joan Hansen, Virum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/066,712

(22) PCT Filed: Sep. 13, 2006
(86) PCT No.: PCT/EP2006/066306

§ 371 Date: Aug. 27, 2008

(30) Foreign Application Priority Data
Sep. 13, 2005 (EP) 05108373.1

(51) Int. Cl. A61M 5/14; A61M 5/32

(52) U.S. Cl. 604/262; 604/174

(57) ABSTRACT

The invention provides a reservoir unit with a reservoir arranged within a housing. The reservoir defines an interior adapted to contain a translucent fluid drug, and comprises a first transparent area. A second area is associated with the reservoir and arranged substantially opposite the first area, the second area comprising a visually non-uniform portion. The housing comprises an inspection portion allowing a user to inspect a portion of the reservoir through the first area with the second area serving as a background, the second area thereby serving as an aid for evaluating a condition of a drug contained in the reservoir, e.g. fibrillated insulin.

The present invention generally relates to a reservoir containing or being adapted to contain a fluid drug, or a device comprising such a reservoir, the reservoir or device comprising means allowing a condition of the drug to be checked, for example, but not restricted to, fibrillation of insulin.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a hollow infusion needle, as well as expelling means for expelling a drug out of the reservoir and through the skin of the subject via the hollow needle. The delivery device may be adapted for discrete use, i.e. injection of an amount of a drug a given number of times during the day, or they may be adapted for continuous or quasi-continuous delivery of drug through a permanent fluid connection between the delivery device and the patient. The former type of device is often referred to as a pen device and the latter type is often termed an infusion pump. A “pen” is typically a mechanical pen-formed device, however, it may have any desirable configuration just as it may comprise a motor for assisted injection of drug.

Basically, infusion pumps can be divided into two classes. The first class comprises durable infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals. Examples of this type of pump are shown in U.S. Pat. Nos. 4,562,751 and 4,685,903 hereby incorporated by reference.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniencies, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also know as a bleeding hole pump)), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference.

The disposable pumps generally comprises a skin-contacting mounting surface adapted for application to the skin of a subject by adhesive means, and with the infusion needle arranged such that in a situation of use it projects from the mounting surface to thereby penetrate the skin of the subject, whereby the place where the needle penetrates the skin is covered while the appliance is in use. When a fluid drug is supplied to a user, it is important that the user can visually inspect the drug to make sure that the drug is not crystallised or polymerised due to e.g. self association or penetration, or that any other visually detectable change of the drug has occurred, such as oxidation of the active drug. For insulin such visual changes are often referred to as “fibrillation”. Even weak degrees of fibrillation can be critical for a patient, as it can potentially cause allergy to insulin and change the time-profile for the insulin. In practice, however, it is relatively difficult to observe weak fibrillation even in a cartridge under good lighting conditions. U.S. Pat. No. 6,251,098 discloses a fluid container for a pump, wherein an embossed area is arranged below a flexible reservoir, the embossed area becoming visible when the reservoir has been emptied.

Having regard to the above-identified prior art devices, it is an object of the present invention to provide a reservoir for a fluid drug, or a device comprising such a reservoir, with means aiding a user to check the condition of the drug, e.g. to check insulin for fibrillation.

### DISCLOSURE OF ASPECTS OF THE INVENTION

In the disclosure of the present invention, embodiments and aspects will be described which will address one or more of the above objects or which will address objects apparent from the below disclosure as well as from the description of exemplary embodiments.

Thus, in a first aspect a reservoir unit is provided comprising housing, and a reservoir arranged at least partially within the housing. The reservoir defines an interior adapted to contain a translucent fluid drug, the reservoir comprising a first transparent area. A second area is associated with the reservoir and arranged substantially opposite the first area, the second area comprising a visually non-uniform portion. The housing comprises an inspection portion allowing a user or device to inspect at least a portion of the reservoir through the first area with the second area serving as a background, the second area serving as an aid for evaluating a condition of a drug contained in the reservoir. The reservoir unit may be prefilled comprising a prefilled reservoir with a fluid drug. By a prefilled reservoir is indicated a reservoir which is supplied to the user in a filled condition and which is not intended to be refilled by the user. By a prefilled reservoir unit is indicated a reservoir unit which is supplied to the user with a prefilled reservoir. The prefilled reservoir may be sealed within the housing of the reservoir unit preventing the user from exchanging or refilling the reservoir.

The visually non-uniform portion may have any desirable configuration which can be found to provide an aid in evaluating a condition of the drug. The “non-uniformity” may e.g. be provided by a regular or irregular graphic pattern or marking, or by a physical surface configuration, marking or pattern. Thus, an actual pattern may vary in accordance with e.g. the type of drug contained in the reservoir, the conditions under which the drug is observed (e.g. the kind of light used), as well as the type of observer who is to evaluate the condition, e.g. the naked eye of a user or a device relative to which the reservoir unit is placed. The pattern may be formed by two or more colours (including black and white as well as shades of the same colour). In the context of the present disclosure mostly the term pattern is used to indicate a visually non-uniform surface portion.

The reservoir may be arranged fully or partly within the housing just as it may be permanently (i.e. not adapted to be exchanged by the user) or mountable arranged within the housing, e.g. a cartridge comprising no evaluation pattern may be arranged in a housing comprising a pattern.

Depending on the configuration of the reservoir, the inspection portion may comprise an opening or a transparent area. For example, for a reservoir in the form of a glass cartridge, the glass per se would form a transparent barrier which could be observed through an opening in the housing, whereas it may be appropriate to protect a flexible reservoir with an additional transparent barrier, e.g. a window in the housing.

The patterned area may be provided by a portion of the reservoir, e.g. the surface pattern may be arranged on an interior surface of the reservoir, or the patterned area may be arranged on an exterior surface of a transparent area. Alternatively the reservoir may comprise a transparent area arranged opposite the first area, the non-uniform surface pattern being arranged on a member external to the reservoir arranged in the vicinity thereof.

In embodiments of the invention the first transparent area provides substantially the only visual access to the reservoir. For example, a reservoir is arranged within a housing having an opening or transparent portion positioned over the first transparent area, the remaining portion of the housing substantially preventing inspection of the interior of the housing.

The reservoir may comprise first and second flexible foil portions sealed together to form an enclosed cavity for containing the fluid, the reservoir having a pouch-like configuration. The reservoir may be initially sealed, the inspection portion and the first and second areas allowing a user to inspect at least a portion of the contained drug in the sealed condition.

The reservoir unit may be provided with a fluid outlet and an expelling assembly adapted for cooperation with the reservoir to expel fluid drug out of the reservoir and through the fluid outlet. The term outlet is used to denote a structure which will serve as an outlet during actual delivery of drug. In other words, the outlet may be closed when not actually used. For example, the outlet may be in the form of a needle-penetratable septum which will be closed until a needle is arranged there through. The outlet may also be provided with a valve which will close the outlet until the delivery expelling means is actuated.

Such a unit may further comprise, or be adapted to cooperate with, a transcutaneous device (e.g. a needle, a soft cannula, a micro needle array, a traditional infusion set or non-invasive transdermal means, projecting from or arranged on a lower surface of a skin-mountable device in a situation of use). The unit may also comprise a mounting surface adapted for application to the skin of the subject, wherein the expelling assembly, in a situation of use, is adapted for expelling drug out of the reservoir and through the skin of the subject via the transcutaneous device.

In an embodiment of the invention a medical device is provided, comprising a transcutaneous device unit and a reservoir unit as described above, the transcutaneous device unit comprising a transcutaneous device and a mounting surface adapted for application to the skin of the subject, wherein the expelling assembly is adapted for cooperation with the reservoir to expel fluid drug out of the reservoir and through the skin of the subject via the transcutaneous device, and wherein the transcutaneous device unit and the reservoir unit are adapted to be secured to each other in a situation of use. The medical device may be adapted to allow inspection of the reservoir only when the reservoir unit is detached from the transcutaneous device unit.

The principles of the present invention may also be implemented in a “pen” type delivery device, such a device comprising a dose setting member moveable to a selected set position representing a set dose of drug to be delivered, and a user actuation member allowing the set dose to be expelled. The expelling means may be purely mechanical or a motor may be used to drive the expelling means.

In a further aspect a reservoir defining an interior containing a translucent fluid drug is provided, comprising a first transparent area, and a second area associated with the reservoir and arranged substantially opposite the first area, the second area comprising a non-uniform surface pattern, whereby a user can inspect at least a portion of the contained drug through the first area with the second area serving as a background, the second area serving as an aid for evaluating a condition of the contained drug. As for the reservoir unit, the reservoir may comprise first and second flexible foil portions sealed together to form an enclosed cavity for containing the fluid, the reservoir having a pouch-like configuration.

The patterned area may be provided by a portion of the reservoir, e.g. the surface pattern may be arranged on an interior surface of the reservoir, or the patterned area may be arranged on an exterior surface of a transparent area. The reservoir may be provided in combination with a further member, the combination comprising a further transparent area arranged substantially opposite the first area, the non-uniform surface pattern being arranged on the member, the member being arranged in the vicinity of the exterior surface of the further transparent area.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject. Further, the term needle (when not otherwise specified) defines a piercing member adapted to penetrate the skin of a subject.

### BRIEF DESCRIPTION OF THE DRAWINGS

In the following the invention will be further described with references to the drawings, wherein

FIG. 1A shows in perspective view an embodiment of a modular drug delivery device,

FIG. 1B shows a side view of an assembled drug delivery device,

FIG. 2 shows in an exploded perspective view a reservoir unit,

FIG. 3 shows in partial a reservoir unit with a portion of the housing removed,

FIG. 4 shows an example of a visually non-uniform pattern,

FIGS. 5A-5C show in cross-section schematic representations of reservoir units comprising a reservoir and a visual inspection aid, and

FIG. 6 shows test results from an inspection study.

In the figures like structures are identified by like reference numerals.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

Firstly, with reference to FIG. 1 an embodiment of a modular drug delivery device will be described. The delivery device is shown as an example of a type of device in which one or more aspects of the present invention advantageously may be implemented, however, aspects of the present invention may be used in combination with any relevant drug delivery device in which it is desirable to check a characteristic of the drug.

The transcutaneous device unit 2 comprises a transcutaneous device in the form of a needle and will thus in the following be termed a needle unit, however, it the context of the present invention, it represents any transcutaneous device that may be used for drug delivery.

More specifically, FIG. 1A shows a perspective view of medical device in the form of a modular skin-mountable drug delivery device 1 comprising a patch-like needle unit 2 and a reservoir unit 5. When supplied to the user each of the units are preferably enclosed in its own sealed package (not shown).

The needle unit comprises a base portion 10 with a lower mounting surface adapted for application to the skin of a user, and a housing portion 20 in which a hollow infusion needle (not shown) is arranged. The needle comprises a distal portion adapted to penetrate the skin of a user, and a proximal portion adapted to be arranged in fluid communication with the reservoir unit. The distal portion of the needle is moveable between an initial position in which the distal end is retracted relative to the mounting surface, and an extended position in which it projects relative to the mounting surface. Further, the needle is moveable between the extended position in which the distal end projects relative to the mounting surface, and a retracted position in which the distal end is retracted relative to the mounting surface. The needle unit further comprises user-gripable actuation means in the form of strip-members 21, 22 for actuating respectively retracting the needle. The housing further comprises user-actuatable male coupling means 40 in the form of a pair of resiliently arranged hook members adapted to cooperate with corresponding female coupling means on the reservoir unit, this allowing the reservoir unit to be releasable secured to the needle unit in the situation of use. The base portion comprises a relatively rigid upper portion 11 attached to a more flexible adhesive sheet member 12 having a lower adhesive surface providing the mounting surface per se, the adhesive surface being supplied with a peelable protective sheet. The base portion also comprises a ridge member 13 adapted to engage a corresponding groove on the reservoir unit.

The reservoir unit 5 comprises a pre-filled reservoir containing a liquid drug formulation (e.g. insulin) and expelling means in the form of an electronically controlled pump for expelling the drug from the reservoir through the needle in a situation of use. The reservoir unit has a generally flat lower surface adapted to be mounted onto the upper surface of the base portion, and comprises a protruding portion 50 adapted to be received in a corresponding cavity of the housing portion 20 as well as female coupling means 51 adapted to engage the corresponding hook members 31 on the needle unit. The protruding portion provides the interface between the two units and comprises a pump outlet and contact means (not shown) allowing the pump to be started as the two units are assembled. The lower surface also comprises a window (not to be seen) allowing the user to visually control the contents of the reservoir, however, such a window may also be arranged on an upper free surface of the reservoir unit.

FIG. 1B shows the reservoir and needle units in an assembled state with a needle 30 protruding from the lower surface thereof.

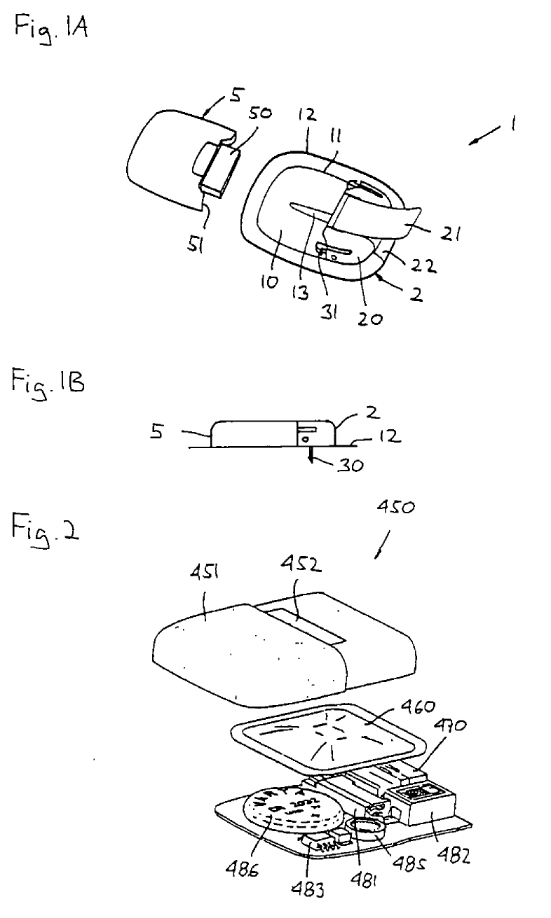

With reference to FIG. 2 an embodiment of a reservoir unit 450 of a type suitable to be used with the above described needle unit is shown or which may comprise the reservoir/pump portion of a unitary device, the reservoir unit comprising a housing 451 in which a flexible foil reservoir 460, a pump unit 470 in the form of a mechanically actuated membrane pump, and control and actuation means therefore are arranged. The reservoir may be connected to the pump when delivered to the user, or a fluid communication between the reservoir and the pump may be established just prior to use, e.g. when the reservoir unit is connected to a needle unit for the first time. The housing comprises a display window 452 which may be used to provide information to the user, e.g. the amount of drug left in the reservoir or information in respect of a malfunction condition, and/or allow the user to visually inspect the contents of the reservoir. The control and actuation means comprises a pump actuating member in the form of a lever and piston arrangement 481 driven by a coil actuator 482, a microprocessor 483 for controlling the different functions of the reservoir unit, signal generating means 485 for generating an audible and/or tactile signal, and an energy source 486. The different components are arranged on a printed circuit board (PCB) 480. In the shown embodiment the reservoir is arranged above the PCB, however, alternatively it may be arranged below the PCB in combination with an inspection window arranged on a lower surface of the reservoir unit. Further details and embodiments in respect of a modular drug delivery device of the type shown in FIGS. 1 and 2 are disclosed in applicant's application WO 2005/039673 which is hereby incorporated in its entirety by reference.

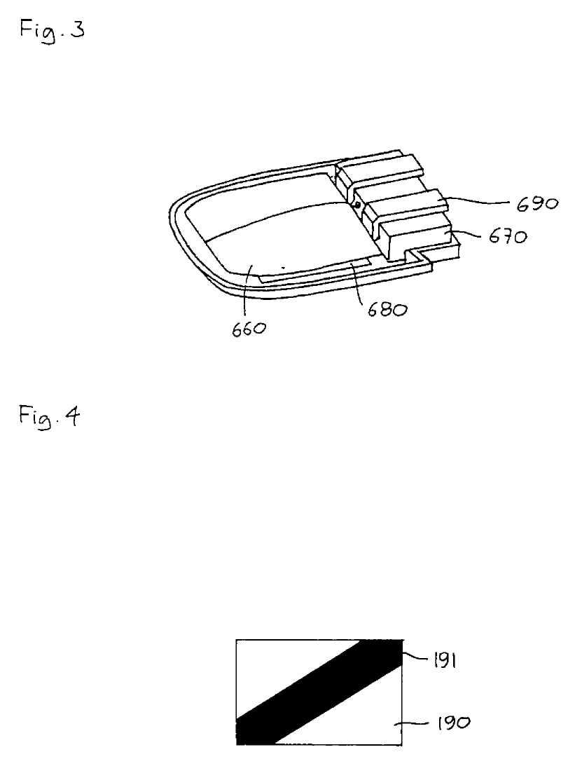

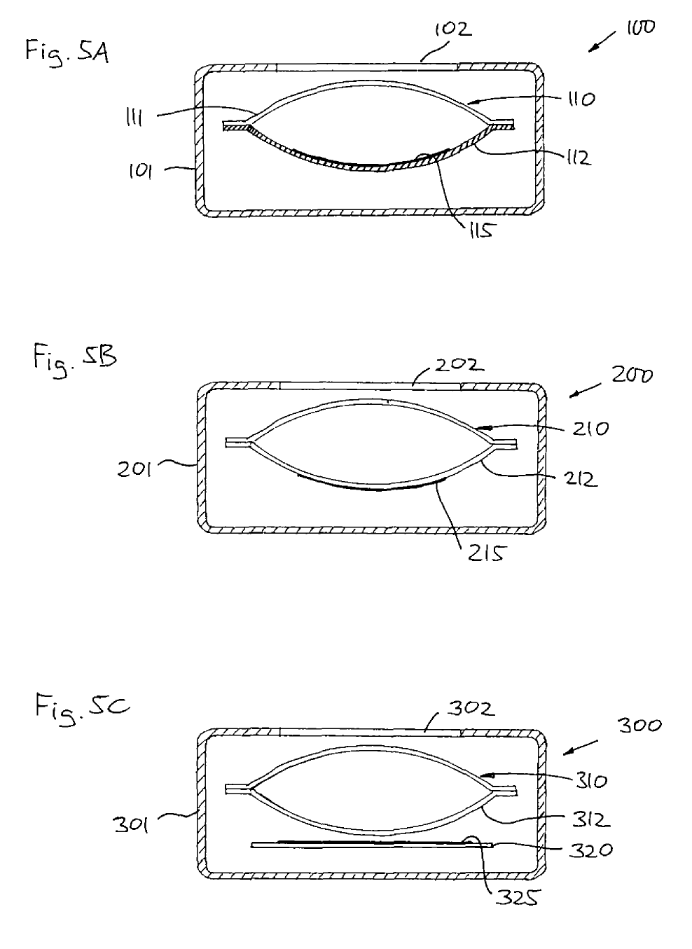

FIG. 3 shows a reservoir unit 650 with a portion of the housing removed. The reservoir unit comprises a pump unit 670, a flexible foil reservoir 660, and two light conductors 690 which may be useful in combination with embodiments of the present invention. The reservoir comprises first and second flexible foil members sealed together at three edges to form a reservoir having a pouch-like configuration defining a general plane, the reservoir having a rounded edge portion. The lower surface of the reservoir unit is adapted to face towards a skin surface in a condition of use and is provided with a transparent area 680 allowing a user to inspect a portion of the reservoir to thereby detect a transmission characteristic of the light through the drug. Corresponding to the FIG. 2 embodiment, the pump unit further comprises a PCB (not shown) with additional components.

Each light conductor comprises a straight inlet portion with a light inlet end, and a straight outlet portion with a light outlet end, the two portions being arranged at an angle relative to each other. The inlet end may form a portion of the outer surface of the reservoir unit just as a light conductor may be formed integrally with other elements, e.g. a housing portion. Between the two portions and at the outlet end facets are provided for conducting light between the portions and out through the outlet end and into the reservoir in a direction substantially corresponding to the general plane of the reservoir. Optical silicone may be used to bridge a gap between the light conductor and the reservoir. In a situation of use the user will orient the light inlet towards a light source, e.g. a lamp, whereby light is conducted to the reservoir allowing the user to inspect the contents through a window in the housing (not shown) arranged on a surface of the reservoir unit or a corresponding device. Alternatively, the reservoir unit may be placed in a detection device which will conduct light via the light conductor into the reservoir and detect a property via the window.

With reference to schematic FIGS. 5A-5C three embodiments of a reservoir unit comprising an aid for evaluating a condition of a drug contained in a reservoir housed within the reservoir unit will be described. The reservoir units are of the type disclosed with reference to FIG. 2, however, for illustrative purposes only the housing and the reservoir are shown.

As shown in FIG. 5A the reservoir unit 100 comprises a sealed housing 101 having a trans-parent window portion 102 allowing the interior of the housing to be visually inspected through the window, the remaining portion of the housing being substantially non-transparent. Within the housing is arranged a reservoir 110 prefilled with a translucent drug and comprising first and second flexible foil portions 111, 112 sealed together to form an enclosed cavity for containing the fluid, the reservoir having a pouch-like configuration. The first foil portion is transparent thus allowing a user or device to inspect at least a portion of the contained drug, the second foil portion being non-transparent with a graphic pattern 115 arranged on the inner surface thereof serving as an aid for evaluating a condition of the drug contained in the reservoir when inspected through the window 102 and the first foil portion 111. Indeed, the pattern may alternatively be arranged on a transparent foil.

The embodiment 200 of FIG. 5B substantially corresponds to the embodiment of FIG. 5A, however, the second foil portion 212 is transparent and the surface pattern is arranged on the exterior surface thereof.

Also the embodiment 300 of FIG. 5C substantially corresponds to the embodiment of FIG. 5A, however, the second foil portion 312 is transparent and the surface pattern 325 is arranged on a member 320 external to the reservoir, the member being arranged in the vicinity of the exterior surface of the second foil portion. The external member may serve primarily for the purpose of carrying the pattern, e.g. a piece of paper, or the external member may serve primarily other purposes, e.g. a rear surface of a PCB or a portion of the housing 301.

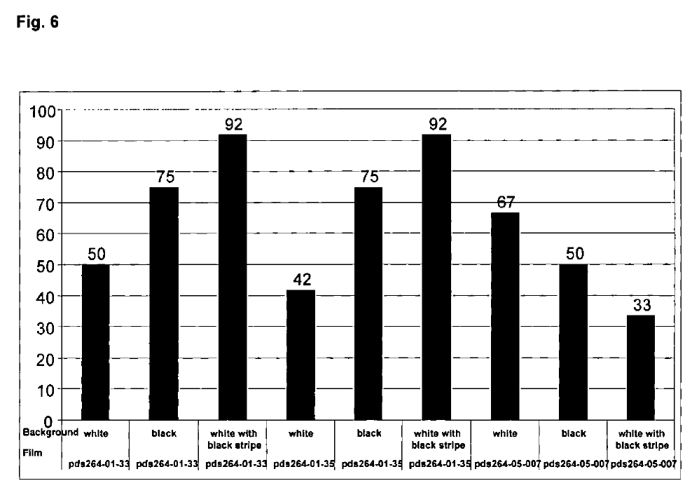

Example: An inspectability study was conducted on a device of the general type shown in FIG. 5C, where it was tested if it was possible to distinguish between a reservoir filled with water and a reservoir filled with a standard test solution. According to Ph. Eur. a plastic container for primary packaging of pharmaceutics should fulfill the demands for clarity described in Ph. Eur. 5.0 Chapter 3.2.2.1, i.e. it should be possible to differentiate between a container comprising water and a formazin suspension sample made in accordance with the Ph. Eur. Chapter 2.2.1. In a final product the reservoir will be placed inside a housing and visual inspection will be possible through a window. A study was set up in order to clarify which reservoir foils could be used and to what extend the inspection background would influence the inspection results. Three films were used having a clarity/transmission compared to water as indicated in the parenthesis: (1) PDS264-01-033: PE/tie/PCTFE/tie/PE (89%), (2) PDS264-01-035: PE/tie/PCTFE/tie/PE (96%), and (3) PDS264-05-007: PETP-AlOx/OPA/PP (86%). The inspection backgrounds were made of paper with the size of 30 times 30 mm. They were black, white or white with a black stripe. The inspection portion of the housings was made from a clear acrylic polymer securing the inspectability. 12 persons participated in the study. A given person was handed two pumps to be inspected at a time in which only the media differed. The person was asked to point out (of the two handed samples) the sample with the most unclear content. 50% correct inspections are the same as random and are therefore considered as not complying with the demands for clarity in Ph. Eur. It is observed from FIG. 6 that two samples obtained 92% correct inspections. Both samples had a white background with a black stripe. From FIG. 1 the following is observed: (i) Both PDS264-01-033 and PDS264-01-035 would be suitable as reservoir film with regards to clarity. (ii) The best inspection background can bring the samples from 50% to 92% correct inspection. The best background was “white with black stripe”. (iii) The samples reaching 92% can be considered very good suggestions for a final product complying with Ph. Eur. The films had different measured clarities, but when formed into reservoirs and filled with media, only the most unclear film did not perform satisfactory. As can be seen, the background had a big impact on the ability to distinguish the formazin suspension from water.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification. For example, in the embodiments of FIGS. 5A-5C the reservoir may be in the form of a traditional cylindrical glass cartridge.

1. A reservoir unit (100, 450) comprising:
a housing (101, 451),
a reservoir (110, 460) arranged at least partially within the housing, the reservoir defining an interior containing a translucent fluid drug, the reservoir comprising a first transparent area (110),
a second area associated with the reservoir and arranged substantially opposite the first area, the second area comprising a visually non-uniform surface portion (115),
wherein the housing comprises an inspection portion (102, 452) allowing a user to inspect at least a portion of the contained drug through the first area with the second area serving as a background, the second area serving as an aid for evaluating a condition of the drug contained in the reservoir.

2. A reservoir unit as in claim 1, wherein the reservoir is arranged within the housing.

3. A reservoir unit as in claim 2, wherein the reservoir is permanently arranged within the housing.

4. A reservoir unit as in claim 1, wherein the inspection portion comprises an opening or a transparent area (102).

5. A reservoir unit as in claim 4, wherein the remaining portion of the housing substantially prevents visual inspection of the reservoir.

6. A reservoir unit as in claim 1, wherein the second area is provided by a portion (112) of the reservoir, the visually non-uniform portion (115) being arranged on the interior surface thereof.

7. A reservoir unit as claim 1, wherein the second area is provided by a transparent portion (212) of the reservoir, the visually non-uniform surface portion (215) being arranged on the exterior surface thereof.

8. A reservoir unit as in claim 1, wherein the reservoir comprises a further transparent area (312) arranged substantially opposite the first area, the visually non-uniform portion (325) being arranged on a member (320) external to the reservoir, the member being arranged in the vicinity of the exterior surface of the further transparent area.

9. A reservoir unit as in claim 1, wherein the reservoir comprises first and second flexible foil portions (111, 112) sealed together to form an enclosed cavity (110) for containing the fluid, the reservoir having a pouch-like configuration.

10. A reservoir unit as in claim 1, wherein the reservoir is provided in an initially sealed condition, the inspection portion and the first and second areas allowing a user to inspect at least a portion of the contained drug in the sealed condition.

11. A reservoir unit as in claim 1, further comprising a fluid outlet and an expelling assembly (470) adapted for cooperation with the reservoir to expel fluid drug out of the reservoir and through the fluid outlet.

12. A reservoir unit as in claim 11, further comprising:
a transcutaneous device adapted to penetrate the skin of a subject, and
a mounting surface adapted for application to the skin of the subject,
wherein the expelling assembly, in a situation of use, is adapted for expelling drug out of the reservoir and through the skin of the subject via the transcutaneous device.

13. A medical device, comprising a transcutaneous device unit (2) and a reservoir unit (5) as in claim 11, the transcutaneous device unit comprising:
a transcutaneous device,
a mounting surface adapted for application to the skin of the subject, wherein the expelling assembly is adapted for cooperation with the reservoir to expel fluid drug out of the reservoir and through the skin of the subject via the transcutaneous device,
wherein the transcutaneous device unit and the reservoir unit are adapted to be secured to each other in a situation of use.

14. A medical device as in claim 13, wherein the inspection portion (680) allows inspection of the reservoir only when the reservoir unit is detached from the transcutaneous device unit.
